(19) United States
(12) Patent Application Publication
(10) Pub. No.: US 2024/0000802 A1
(43) Pub. Date: Jan. 4, 2024

(54) DIFLUPREDNATE FOR REDUCING THE ADVERSE EFFECTS OF OCULAR INFLAMMATION

(71) Applicant: SUN PHARMA ADVANCED RESEARCH COMPANY LIMITED, Mumbai, Maharashtra (IN)

(72) Inventors: Ajay Jaysingh KHOPADE, Vadodara, Gujarat (IN); Arindam HALDER, Vadodara, Gujarat (IN)

(21) Appl. No.: 18/255,207

(22) PCT Filed: Dec. 3, 2021
(86) PCT No.: PCT/IB2021/061289

§ 371 Date: May 31, 2023

(30) Foreign Application Priority Data
Dec. 3, 2020 (IN) 202021052718
Dec. 3, 2021 (WO) PCT/1B2021/61289
Dec. 3, 2021 (WO) PCT/IB2021/61289

(51) Int. Cl. A61K 31/573; A61K 9/00; A61P 27/02; A61P 29/00

(52) U.S. Cl. CPC A61K 31/573; A61K 9/0048; A61P 27/02; A61P 29/00

(57) ABSTRACT

The present invention provides an aqueous solution comprising difluprednate as the sole active ingredient, wherein the aqueous solution comprises 0.02% to 0.04% w/v difluprednate and an aqueous vehicle, wherein the aqueous solution is free of oil and wherein the aqueous solution is administered twice-a-day for 7 to 21 days to the subject and a method of reducing an adverse effect associated with an inflammatory disorder of eye in a subject in need thereof, using said aqueous solution.

### FIELD OF THE INVENTION

The present disclosure relates to a method of reducing the adverse effects of an inflammatory disorder of the eye in a subject in need thereof, said method comprising administering into the eye of the subject an ophthalmic aqueous solution of difluprednate.

### BACKGROUND OF THE INVENTION

Difluprednate is an anti-inflammatory corticosteroid drug represented by formula I below.

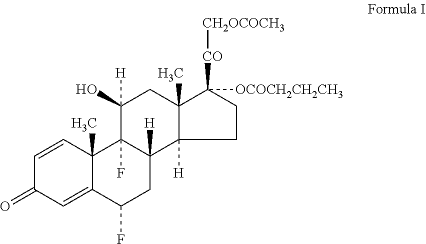

Difluprednate, a steroidal drug is practically insoluble in aqueous vehicle. The currently marketed formulation of difluprednate is an emulsion dosage form of difluprednate, approved and marketed in the United States under the brand name of DUREZOL® (Novartis, East Hanover, NJ). DUREZOL® is an ophthalmic emulsion formulation of difluprednate which comprises 0.05% w/v difluprednate emulsified between castor oil and water. It is not a clear aqueous solution. The U.S. Pat. No. 6,114,319 (herein after referred to as the '319 patent) is listed in the “Orange Book: Approved Drug Products with Therapeutic Equivalence Evaluations” against DUREZOL® product and describes an emulsion formulation of difluprednate which contains an oil and an emulsifier. DUREZOL® emulsion formulation is indicated for the treatment of inflammation and pain associated with ocular surgery and endogenous anterior uveitis when administered four times a day. As the emulsion needs to be instilled four times-a-day, there are high chances of patient non-compliance and missing a dose. The prior art formulations of difluprednate does not provide prolonged action. Further, it has been reported and also noted in the approved label of DUREZOL® that the most common adverse reactions in subjects exposed to DUREZOL® (occurring in 5-10% of subjects), include blurred vision, eye irritation, eye pain, headache, increased TOP, iritis, limbal and conjunctival hyperemia, and punctate keratitis. Thus, there lies a need for a formulation of difluprednate that is devoid of these side effects and which addresses the existing drawbacks.

### SUMMARY OF THE INVENTION

The present disclosure provides an aqueous solution comprising a. therapeutically effective concentration of difluprednate, a crystal growth inhibitor and pharmaceutically acceptable amounts of a solubilizer comprising a mixture of: i) quaternary ammonium compound and ii) polyethoxylated castor oil, in an aqueous vehicle wherein the crystal growth inhibitor is polyvinyl alcohol or its derivatives.

The present disclosure provides in another aspect, a method of treating inflammatory disorder of the eye, said method comprising administering into the eye of a person in need thereof, an aqueous solution comprising difluprednate as the sole active ingredient at a concentration of about 0.02% to 0.04% weight by volume, wherein the solution is free of oil and wherein the solution is administered twice-a-day, and wherein the subject has not received rescue medication and after the treatment the subject had an anterior chamber cell (ACC) grade of 0 and a pain score of 0 on the visual analog scale (VAS) post ocular surgery.

In some embodiments, the disclosure provides a method of reducing an adverse effect associated with an inflammatory disorder of eye in a subject in need thereof, said method comprising administering to the eye of the subject an aqueous solution comprising difluprednate as the sole active ingredient, wherein the aqueous solution comprises 0.02% to 0.04% w/v difluprednate and an aqueous vehicle, wherein the aqueous solution is free of oil and wherein the aqueous solution is administered twice-a-day for 7 to 21 days to the subject.

In some embodiments, the disclosure provides a method of reducing an adverse effect associated with an inflammatory disorder of eye in a population of subjects in need thereof, said method comprising administering to the eyes of the population of subject an aqueous solution comprising difluprednate as the sole active ingredient, wherein the aqueous solution comprises 0.02% to 0.04% w/v difluprednate and an aqueous vehicle, wherein the aqueous solution is free of oil and wherein the aqueous solution is administered twice-a-day for 7 to 21 days to the population of subjects.

### DETAILED DESCRIPTION OF THE INVENTION

The “aqueous solution” as stated herein, is a solution of difluprednate in aqueous vehicle, wherein difluprednate is in solubilized form and not in particulate form, either microparticulate or nanoparticulate or in micellar form.

The term ‘crystal growth inhibitor’ as used herein means the additional excipients that prevent the difluprednate from being precipitated or crystallized out from the aqueous vehicle. The screening for inhibitory effect of crystal growth of difluprednate may be carried out by physical observation as well as by determining the clarity of the aqueous solution, immediately upon formulating or on storage. The solutions show percentage transmission greater than 90%, e.g. greater than 95%. When light is allowed to pass through the ophthalmic solution, the percentage of incident light which is transmitted through the solution is referred to as “percent transmission”. Generally, the percentage transmission is determined at a wavelength of about 650 nm, but any other suitable wavelength may be selected for determining the clarity of the solution. The aqueous solution of the present disclosure show percent transmission greater than 90%, e.g., greater than 95%, even greater than 99%. The aqueous solution remains clear and free from particles, crystals or precipitate, upon long term storage at temperatures between 2° C. to 30° C. for a period of 6 months or more.

The aqueous solution of the present disclosure is free of oil. The term ‘oil’ as used herein refers to oils which are hydrophobic compounds. The examples of the ‘oil’ include, but are not limited to triglycerides such as, castor oil, peanut oil, arachis oil, mineral oil and the like. The term ‘oil’ does not include amphiphilic compounds or surfactants obtained by derivatising oil with a hydrophilic entity such as for example polyethoxylated castor oil.

The ophthalmic aqueous solution of the present disclosure comprises a. therapeutically effective concentration of difluprednate, a crystal growth inhibitor and pharmaceutically acceptable amounts of a solubilizer comprising a mixture of: i) quaternary ammonium compound and ii) polyethoxylated castor oil, in an aqueous vehicle wherein the crystal growth inhibitor is polyvinyl alcohol or its derivatives.

The ophthalmic aqueous solution used according to the present disclosure comprises difluprednate as the sole therapeutically active ingredient. The word ‘difluprednate’ as used herein includes prodrugs of difluprednate wherein the hydroxyl group in difluprednate is converted to a labile ester or an amide. In one embodiment, the ophthalmic aqueous solution according to the present disclosure does not include povidone-iodine or any other active ingredient and always contains a sole active ingredient. The concentration (% weight by volume) of difluprednate is expressed in terms of difluprednate base. It is present at a concentration that ranges from about to 0.07% weight by volume, e.g., from about 0.02% to 0.045% weight by volume, such as for example 0.025, 0.03, 0.035, 0.036, 0.037, 0.038, 0.039 or 0.04%, 0.042%, 0.043% weight by volume, or from about 0.02% to 0.04% weight by volume.

The ophthalmic aqueous solution according to the present disclosure comprises a solubilizer which is a mixture of a quaternary ammonium compound and, polyethoxylated castor oil. It was found that when an individual solubilizer i.e. quaternary ammonium compound alone or polyethoxylated castor oil alone was used, the attempts to solubilize difluprednate were not successful and difluprednate precipitated from the solution, either immediately or upon storage. Surprisingly, when the mixture of these two category of solubilizers was used, clear aqueous solution was obtained i.e. there was no precipitation of difluprednate upon preparation or on storage.

The quaternary ammonium compound is selected from, but not limited to, benzalkonium chloride, myristyl gamma picolinium chloride, benzethonium chloride, benzododecinium bromide, cetalkonium chloride, cetylpyridinium chloride, cetrimonium, tetraethylammonium bromide, polyhexamethylene biguanide, oleyl amine and the like. In some embodiments, the quaternary ammonium compound is selected from benzalkonium chloride and myristyl gamma picolinium chloride. The quaternary ammonium compound is used in the ophthalmic solution in amounts ranging from about 0.0002% to 0.1% weight by volume, e.g., from about 0.002% to about 0.08% weight by volume, such as for example 0.003, 0.004, 0.005, 0.006, 0.007, 0.008, 0.009, 0.01, 0.02, 0.04, 0.05, 0.06 or 0.07% weight by volume. According to one particular embodiment, the quaternary ammonium compound is benzalkonium chloride and is present in the ophthalmic aqueous solution in an amount ranging from about 0.0002% to 0.08% weight by volume, e.g., from about 0.0002% to 0.05% weight by volume, or from about 0.005% to 0.05% weight by volume, e.g., from about 0.01% to 0.03% weight by volume.

The polyethoxylated castor oil that is used as a solubilizer according to the present disclosure is also known by other terms like polyoxyl castor oil, or polyoxyethylene castor oil. It is marketed under various tradenames such as Cremophor®, Acconon®, Arlatone®, Eumulgin®, Etocas®, Jeechem®, Hetoxide®, Nikkol®, and Croduret®. The polyethoxylated castor oils or polyoxyethylene castor oil derivatives that may be used in the ophthalmic aqueous solutions of the present disclosure are described in “Handbook of Pharmaceutical Excipients”, fifth edition, 2006, page 572-578. In some embodiments, the ophthalmic aqueous solution of the present disclosure comprises polyoxyl 35 castor oil, marketed under the tradename Cremophor® EL by BASF Corp., polyoxyl 40 castor oil marketed under the tradename Croduret®40 or Etocas®40, polyoxyl 60 castor oil marketed under the tradename Jeechem® CA-60; polyoxyl 15 castor oil marketed under the tradename Jeechem®CA-15 or Acconon®CA-15. In some embodiments, the polyethoxylated castor oils are used in the ophthalmic aqueous solution of the present disclosure in pharmaceutically acceptable amounts. In some embodiments, the pharmaceutically acceptable amount of polyethoxylated castor oil′ ranges from about 1.0% to 10.0% weight by volume. In some embodiments, the polyethoxylated castor oil is present in the ophthalmic aqueous solution in an amount ranging from about 1.5% to 6.0% w/v, such as for example 2.0, 2.5, 3.0, 3.5, 4.0, 4.5, 5.0, or 5.5% weight by volume of the solution. According to one particular embodiment, the ‘polyethoxylated castor oil’ is polyoxyl 35 castor oil and is present in the ophthalmic aqueous solution in pharmaceutically acceptable amount ranging from about 3.0% to 6.0% w/v.

The crystal growth inhibitor present in the aqueous solution is polyvinyl alcohol or its derivatives. Without the presence of a crystal growth inhibitor, difluprednate does not remain in solubilized form in an aqueous vehicle and precipitates out upon standing/storage. The derivatives of the polyvinyl alcohol include, but are not limited to, polyvinyl alcohol-polyethylene glycol graft copolymer (marketed under the trade name Kollicoat®), poly (vinyl alcohol co ethylene), polystyrene-polyvinyl alcohol graft co-polymer, polyvinyl alcohol-polyvinylpyrrolidone graft co-polymer, polyvinyl alcohol-lactic acid graft co-polymer, polyvinyl alcohol-carregeenan-graft co-polymer, polyvinyl alcohol-polyether graft copolymer and the like and mixtures thereof. In one specific embodiment, the crystal growth inhibitor is polyvinyl alcohol. In one embodiment, the crystal growth inhibitor ranges from about 0.1% to 5.0% weight by volume, e.g., from about 0.5% to 3.0%, such as for example 0.6, 0.7, 0.8 0.9, 1.0, 1.1, 1.2, 1.3, 1.4, 1.5, 1.6, 1.7, 1.8, 1.9, 2.0, 2.2, 2.4, 2.5, 2.6, 2.7, 2.8 or 2.9% weight by volume of the solution. In another embodiment, the amount of crystal growth inhibitor ranges from about 1% to about 2% weight by volume.

The aqueous solution of the present disclosure may further include other conventional excipients such as surfactants, viscosity increasing agent, preservatives, chelating agents, cosolvents, buffers and so on.

The surfactants used in the present disclosure comprise a mixture of non-ionic, anionic, and cationic surfactants. The non-ionic surfactants are selected from Polyethoxylated castor oil, alkyl ethers such as polyoxyethylene octyl ether, polyoxyethylene lauryl ether, polyoxyethylene stearyl ether and polyoxyethylene oleyl ether; alkyl phenyl ethers such as polyoxyethylene octylphenyl ether and polyoxyethylene nonylphenyl ether; alkylesters such as polyoxyethylene laurate, polyoxyethylene stearate and polyoxyethylene oleate; alkylamines such as polyoxyethylene laurylamino ether, polyoxyethylene stearylamino ether, polyoxyethylene oleylamino ether, polyoxyethylene soybean aminoether and polyoxyethylene beef tallow aminoether; alkylamides such as polyoxyethylene lauric amide, polyoxyethylene stearic amide and polyoxyethyleneoleic amide; vegetable oil ethers such as polyoxyethylene rapeseed oil ether; alkanol amides such as lauric acid diethanol amide, stearic acid diethanol amide and oleic acid diethanol amide; and sorbitan ester ethers such as polyoxyethylene sorbitan monolaurate, polyoxyethylene sorbitan monopalmitate, polyoxyethylene sorbitan monostearate and polyoxyethylene sorbitan monooleate. In some embodiments, the surfactant is polyethoxylated castor oil. In one or more embodiments, the amount of non-ionic surfactant ranges from about 1.5% to about 6.0% weight by volume.

The anionic surfactants are selected from acyl sarcosines or sarcosinates.

The acylated amino acids according to the present disclosure are N-acyl fatty acid derivatives of natural amino acids. These are generally anionic in nature and are soluble in water. The acylated amino acid used in the ophthalmic solution is selected from a group consisting of but not limited to acyl sarcosines or sarcosinates, acyl glutamates, acyl glycinates, acyl aspartates, acyl taurates, acyl malonates or acyl amino-malonates, their salts or mixtures thereof. Generally, the salts include sodium salt, potassium salt, ammonium salt, amine salt, triethanolamine salt and the like.

The acyl sarcosines and their salts are represented by formula II given below:

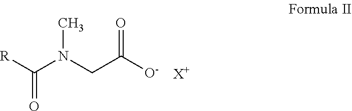

wherein R is a fatty acid group having C₄-C₂₁ carbon atoms;
X represents a salt, for example, a sodium salt, potassium salt, ammonium salt, amine salt, tri-ethanol amine salt and the like.
Non-limiting examples of acyl sarcosines include, but are not limited to, N-lauroyl sarcosine, N-oleoyl sarcosine, N-stearoyl sarcosine, N-myristoyl sarcosine, N-cocoyl sarcosine or their salts such as N-lauroyl sarcosine sodium or sodium N-lauroyl sarcosinate, N-lauroyl sarcosine potassium or potassium N-lauroyl sarcosinate, sodium N-oleoyl sarcosinate, sodium N-stearoyl sarcosinate, sodium N-myristoyl sarcosinate, sodium N-cocoyl sarcosinate, ammonium cocoyl sarcosinate, ammonium lauroyl sarcosinate and the like or mixtures thereof.
Non-limiting examples of acyl glutamates include, but are not limited to, N-lauroyl glutamate, N-oleoyl glutamate, N-stearoyl glutamate, N-myristoyl glutamate or salts thereof such as, for example, mono-sodium N-lauroyl glutamate; potassium N-lauroyl glutamate and the like or mixtures thereof.
Non-limiting examples of N-acyl glycinates include, but are not limited to, N-lauroyl glycinate, N-oleoyl glycinate, N-stearoyl glycinate, N-myristoyl glycinate or salts thereof. Non-limiting examples of N-acyl aspartate include, but are not limited to, N-lauroyl apartate, N-oleoyl apartate, N-stearoyl aspartate, N-myristoyl aspartate or salts thereof. Non-limiting examples of N-acyl taurates include, but are not limited to, N-lauroyl taurate, N-oleoyl taurate, N-stearoyl taurate, N-myristoyl taurate, N-methyl acyl taurates or salts thereof. Non-limiting examples of acyl aminomalonates include, but are not limited to, N-lauroyl aminomalonate, N-oleoyl aminomalonate, N-stearoyl aminomalonate, N-myristoyl aminomalonate or salts thereof.

In some embodiments, the acylated amino acid is a sarcosine compound, i.e. N-acyl sarcosine or its salt, e.g., N-lauroyl sarcosine or its salt. In a particular embodiment, the acylated amino acid is a sodium salt of N-lauroyl sarcosine, i.e. N-lauroyl sarcosine sodium or sodium N-lauroyl sarcosinate, which is represented by a compound of formula III below:

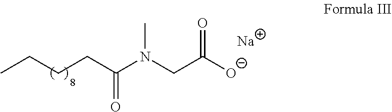

In another embodiment, the acylated amino acid is a sodium salt of N-lauroyl glutamate, i.e. mono sodium N-lauroyl glutamate.

Other anionic surfactants which can be used include, but are not limited to, lactylates, alkylbenzene sulfonate, alkyl sulfate, polyoxyethylene alkyl sulfate, aliphatic α-sulfomethyl ester, α-olefin sulfonic acid, sodium lauryl sulfate, dioctyl sodium sulfosuccinate, the triethanolamine salt of n-acylated polypeptide sodium n-lauryl sarcosinate and triethanol ammonium lauryl sulphate, and sodium butyl naphthylene sulfonate.

In some embodiments, the anionic surfactant is N-lauroyl sarcosine. In one or more embodiments, the amount of an anionic surfactant ranges from about 0.01% to 0.1% w/v, e.g., from about 0.03 to 0.06% w/v, such as for example 0.03, 0.035, 0.04, 0.045, 0.05, 0.06% w/v.

The cationic surfactants are selected from selected from primary, secondary and tertiary highly cationizable and quaternary amines. In a more particular embodiment, the cationic surfactant is selected from oleylamine, stearylamine, benzalkonium chloride, benzethonium chloride, cetylpyridinium chloride, cetyltiridinium bromide, dodecyltrimethylammonium bromide, trimethyltetradecylamonium bromide, hexadecyltrimethylammonium bromide and poloxamines (e.g., Tetronic™) or mixtures thereof. In some embodiments, the cationic surfactant is benzalkonium chloride. In one or more embodiments, the amount of cationic surfactant ranges from about 0.002% to about 0.3% weight by volume.

In some embodiments, the surfactant system comprises a mixture of polyethoxylated castor oil, N-lauroyl sarcosine and benzalkonium chloride. The mixture of non-ionic, cationic and anionic surfactant forms may be mixed in a micellar system in which the water insoluble difluprednate is solubilized.

Absence of any of the compounds such as quaternary ammonium compound (benzalkonium chloride) or polyethoxylated castor oil (Cremophor®) or an acylated amino (N-lauroyl sarcosine), can change the functional properties such as solubilization, stabilization and preservative efficacy of the formulation.

The viscosity increasing agents that can be used include, but are not limited to, carboxymethylcellulose, hydroxypropyl methylcellulose, hydroxyethyl cellulose, hydroxypropyl cellulose, methylcellulose, hydroxyethyl starch, dextran, xanthan gum, sodium alginate, starch, sodium hyaluronate, carbopols, polyvinyl pyrrolidone and the like or mixtures thereof. In some embodiments, the viscosity increasing agent is carboxymethylcellulose. Suitably, in some embodiments, the aqueous solution does not contain a viscosity increasing agent such as polycarbophil and chitosan which have high molecular weight. The viscosity of the aqueous solution according to the present disclosure generally ranges from about 1 cps to 2000 cps, e.g., about 2 cps to 1000 cps, about 1 cps to 300 cps, or from about 2 cps to 200 cps. In some embodiments, the viscosity of the ophthalmic aqueous solution is between about 2 cps to 30 cps. In one embodiment, the present disclosure provides an ophthalmic aqueous solution comprising a therapeutically effective amount of difluprednate as the sole active ingredient and having a viscosity from 2 cps to 200 cps, wherein the solution is effective in treating inflammatory disorder of the eye when administered twice-a-day.

The ophthalmic aqueous solution may further contain one or preservatives, particularly when the dosage form is a multiple dose and not a single dose. The preservatives that may be used include, but are not limited to, benzyl alcohol, cetrimide, chlorhexidine, chlorobutanol, mercurial preservatives like phenylmercuric nitrate, phenylmercuric acetate, thimerosal, phenylethyl alcohol, Polyquad®, stabilized oxy-chlorocomplex, stabilized peroxides and perborates and the like. It is also possible to include safer preservative systems and preservative efficacy enhancers such as edetate disodium, N-lauroyl sarcosine or its sodium salt, boric acid, borates, biguanides like polyhexamethylene biguanide, polyoxyalkylene diamine biguanide or its water-soluble salt; 1,1′-hexamethylene-bis-{5-(4-chlorophenyl)-biguanide}; 1,1′-hexamethylene-bis-{5-(4-fluorophenyl)-biguanide}; (N,N″-bis(2-ethyl hexyl)-3,12-diimino-2,4,11,13-tetraazatetra decanediimidamine; parabens (such as methyl-propyl, isopropyl and butyl-paraben), pyruvates, stabilized oxychloro compounds, sorbic acid/potassium sorbate, metal ions, peroxides, amino acids such as arginine, tromethamine and mixtures thereof. According to another embodiment of the present disclosure, the ophthalmic aqueous solution may be self-preserving.

In one embodiment, the ophthalmic aqueous solution uses a preservative selected from a biguanide, boric acid, N-lauroyl sarcosine or mixtures thereof. In one embodiment, the ophthalmic aqueous solution comprises polyhexamethylene biguanide in an amount ranging from about 0.001% to 0.04% w/v, e.g., from about 0.002% to 0.02% w/v, or, for example, 0.003, 0.004, 0.005, 0.006, 0.007, 0.008, 0.009, 0.01 or 0.02% w/v. In one embodiment, the ophthalmic aqueous solution comprises boric acid in an amount ranging from about 0.05% to 1.5% w/v, e.g., from about 0.1% to 1.0% w/v, such as for example, 0.3, 0.4, 0.5, 0.6, 0.7, 0.8 or 0.9% w/v, or from 0.4% to 0.7% w/v. In one embodiment, the ophthalmic aqueous solution comprises N-lauroyl sarcosine in an amount ranging from about 0.001 to 0.5% w/v, such as for example 0.002, 0.003, 0.004, 0.005, 0.006, 0.007, 0.009, 0.01, 0.02, 0.03, 0.04, 0.05, 0.06, 0.07, 0.08, 009, 0.1, 0.15, 0.2, 0.25, 0.3, 0.35, 0.4 or 0.45% w/v, e.g., from about 0.01 to 0.1% w/v, or from 0.02 to 0.05% w/v. In one particular embodiment, the ophthalmic aqueous solution comprises a mixture of polyhexamethylene biguanide, boric acid and N-lauroyl sarcosine as preservatives. In one specific embodiment, it was found that when benzalkonium chloride was used alone, the solution was not preserved effectively, which was indicated by the results of the preservative efficacy test. When a biguanide was added to the solution, the solution was found to be preserved effectively, i.e., the solution passed the preservative efficacy test as specified in European Pharmacopoeia.

The chelating agents that can be used include, but are not limited to, edetate disodium, ethylenediamine tetracetic acid, edetic acid, disodium edetate dihydrate, diethylenetriamine pentaacetic acid, and the like. A preferred chelating agent is ethylenediamine tertaacetic acid or disodium edetate. In one embodiment, the ophthalmic aqueous solution comprises disodium edetate in an amount ranging from about 0.001% to w/v, such as for example 0.002, 0.003, 0.004, 0.005, 0.006, 0.007, 0.008, 0.009, 0.01, 0.03, 0.04, 0.05, 0.06, 0.07, 0.08, 0.09, 0.1, 0.15, 0.2, 0.25, 0.3, 0.35, 0.4 or 0.45% w/v, e.g., from about 0.01 to 0.1% w/v, or in an amount of 0.03-0.07% w/v.

The pH adjusting agents and/or buffer that may be used are selected from, but not limited to, acetic acid, sodium acetate, tartaric acid, sodium tartrate, citric acid, sodium citrate, hydrochloric acid, sodium hydroxide or mixtures thereof. The osmotic/tonicity adjusting agents that may be used include, but are not limited to, sodium chloride, potassium chloride, sodium bromide, calcium chloride, mannitol, glycerol, sorbitol, propylene glycol, dextrose, sucrose, mannose and the like and mixtures thereof. These solutions are characterized by osmolalities of 250-375 mOsm/kg, or 270-350 mOsm/kg, such as for example 275, 280, 285, 290, 295, 300, 305, 310, 315, 320, 325, 330, 335, 340 or 345 mOsm/kg. Osmolality of the solutions is adjusted by addition of an osmotic/tonicity adjusting agent.

The co-solvents that can be used, include, but are not limited to, glycerol or glycerine, propylene glycol, ethylene glycol, polyethylene glycol, glycofurol and like. The co-solvents, for example, glycerol, may be present in the ophthalmic aqueous solution of the present disclosure in an amount ranging from about 0.5% w/v to about 5.0% w/v, such as for example 1.0, 1.5, 2.0, 2.5, 3.0, 3.5, 4.0, or 4.5% w/v, e.g., from about 1.0% w/v to about 3.0% w/v of the solution.

A representative aqueous solution of the present disclosure comprises the following composition:

| Ingredient/function | Range % weight by volume |
|---|---|
| Difluprednate | 0.02-0.04 |
| A quarternary ammonium compound | 0.0002-0.08 |
| (as solubilizer 1) | |
| Polyethoxylated castor oil | 1.5-6.0 |
| (as solubilizer 2) | |
| Polyvinyl alcohol or its derivative | 0.1-5.0 |
| as a Crystal growth Inhibitor | |
| Aqueous Vehicle | q.s to 100 |

According to various aspects, the ophthalmic aqueous solution according to the present disclosure comprises the following composition:

| Ingredient/function | Range % weight by volume |
|---|---|
| Difluprednate | 0.02-0.04 |
| A quarternary ammonium compound | 0.0002-0.08 |
| (as solubilizer 1) | |
| Polyethoxylated castor oil | 1.5-6.0 |
| (polyoxyl 35 castor oil) | |
| Polyvinyl alcohol or its derivative | 0.1-5.0 |
| as a Crystal growth Inhibitor | |
| Cosolvent | 0.5-5.0 |
| Aqueous Vehicle for e.g., | q.s to 100 |
| Water for injection | |

According to various aspects, the ophthalmic aqueous solution according to the present disclosure comprises the following composition:

| Ingredient/function | Range % weight by volume |
|---|---|
| Difluprednate | 0.02-0.04 |
| Quarternary ammonium compound | 0.0002-0.08 |
| Polyethoxylated castor oil | 1.5-6.0 |
| Polyvinyl alcohol or its derivative | 0.1-5.0 |
| as a Crystal growth Inhibitor | |
| Cosolvent | 0.5-5.0 |
| N-lauroyl sarcosine | 0.01-0.1 |
| Poly hexa methylene biguanide | 0.001-0.04 |
| Other Preservative | 0.1-1.0 |
| Chelating agent | 0.01-0.1 |
| Buffer | 0.001-0.05 |
| Water for Injection | q.s to 100 |

According to various aspects, the ophthalmic aqueous solution according to the present disclosure comprises the following composition:

| Ingredient/function | Range % weight by volume |
|---|---|
| Difluprednate | 0.03-0.04 |
| Quarternary ammonium compound | 0.01-0.05 |
| Polyethoxylated castor oil | 3.0-5.0 |
| Crystal growth Inhibitor, Polyvinyl | 0.5-3.0 |
| alcohol or its derivative | |

A first ophthalmic aqueous solution, according to various aspects, comprises the following composition:

| Ingredient/function | Range % weight by volume |
|---|---|
| Difluprednate | 0.03-0.04 |
| Quarternary ammonium compound | 0.025 |
| N-lauroyl sarcosine | 0.03 |
| Polyethoxylated castor oil | 5.00 |
| Polyvinyl alcohol or its derivative | 1.40 |
| Poly hexa methylene biguanide | 0.005 |
| Other conventional excipients | 0.05 to 2.0 |
| Aqueous Vehicle for e.g., | q.s to 100 |
| Water for Injection | |

A second ophthalmic aqueous solution, according to various aspects, comprises the following composition:

| Ingredient/function | Range % weight by volume |
|---|---|
| Difluprednate | 0.03 |
| Benzalkonium chloride | 0.02 |
| Polyoxyl 35 castor oil (Cremophor ®,) | 4.0 |
| Polyvinyl alcohol or its derivative | 1.4 |
| Other conventional excipients | 0.05 to 2.0 |
| Aqueous Vehicle for e.g., | q.s to 100 |
| Water for Injection | |

The present disclosure provides, in another aspect, a method of treatment of inflammatory disorder of the eye, said method comprising administering into the eye of the subject in need thereof, an aqueous solution comprising difluprednate as the sole active ingredient at a concentration of 0.02% to 0.04% weight by volume in an aqueous vehicle, wherein the solution is free of oil and wherein the solution is administered twice-a-day, and wherein the subject has not received rescue medication and after the treatment the subject had an anterior chamber cell (ACC) grade of 0 and a pain score of 0 on the visual analog scale (VAS) post ocular surgery.

The inflammatory disorder of the eye may be one or more of the pain and inflammation associated with ocular surgery and uveitis. In a particular aspect, the present disclosure provides a method of treating pain and inflammation associated with ocular surgery, said method comprising twice-a-day administration into the eye of a person in need thereof, an aqueous solution comprising difluprednate as the sole active ingredient at a concentration of 0.02% to 0.04% weight by volume in an aqueous vehicle, wherein the solution is free of oil.

Particularly, the present disclosure provides a method of treating pain and inflammation associated with ocular surgery, said method comprising twice-a-day administration into the eye of a person in need thereof, an aqueous solution comprising difluprednate as the sole active ingredient at a concentration of 0.04% weight by volume in an aqueous vehicle, wherein the solution is free of oil and wherein the subject has not received rescue medication and after the treatment the subject had an anterior chamber cell (ACC) grade of 0 and pain score of 0 on the visual analog scale (VAS) post ocular surgery.

Accordingly, some aspects of the present disclosure provide a method of achieving an anterior chamber cell (ACC) grade of 0 in a subject in need thereof, the method comprising administering into the eye of the subject an aqueous solution comprising difluprednate as the sole active ingredient at a concentration of 0.02% to 0.04% weight by volume in an aqueous vehicle, wherein the solution is free of oil, wherein the solution is administered twice-a-day for 7-21 days, and wherein the subject has not received rescue medication. In certain aspects, the subject in need thereof has undergone ocular surgery.

Another aspect of the present disclosure is a method of achieving a pain score of 0 on the visual analog scale (VAS) in a subject in need thereof, the method comprising administering into the eye of the subject an aqueous solution comprising difluprednate as the sole active ingredient at a concentration of 0.02% to 0.04% weight by volume in an aqueous vehicle, wherein the solution is free of oil, wherein the solution is administered twice-a-day for 7-21 days, and wherein the subject has not received rescue medication. In certain aspects, the subject in need thereof has undergone ocular surgery.

In a particular embodiment, the present disclosure provides a method of treating pain and inflammation associated with cataract surgery, said method comprising twice-a-day administration into the eye of a person in need thereof, an aqueous solution comprising difluprednate as the sole active ingredient at a concentration of 0.04% weight by volume in an aqueous vehicle, wherein the solution is free of oil and wherein the subject has not received rescue medication and after the treatment the subject had an anterior chamber cell (ACC) grade of 0 and pain score of 0 on the visual analog scale (VAS) post ocular surgery.

Some embodiments of the present disclosure include a method of reducing inflammation in the anterior chamber of the eye comprising administering into the eye of a subject that has undergone ocular surgery an aqueous solution comprising difluprednate as the sole active ingredient at a concentration of 0.02% to 0.04% weight by volume in an aqueous vehicle, wherein the solution is free of oil, wherein the solution is administered twice-a-day, wherein the subject has not received rescue medication, and wherein after the treatment the subject has an anterior chamber cell (ACC) grade of 0 and a pain score of 0 on the visual analog scale (VAS) post ocular surgery following 15 days of administration. In certain aspects, the ocular surgery is cataract surgery.

Another embodiment of the present disclosure is a method of treatment of inflammatory disorder of eye, the method comprising administering into the eye of a patient in need thereof an aqueous solution comprising difluprednate as the sole active ingredient at a concentration of 0.02% to 0.04% w/v in an aqueous vehicle, wherein the solution is free of oil and wherein the solution is administered twice-a-day for 14 days, wherein the patient has not received rescue medication and after the treatment the patient has an anterior chamber cell grade of 0. In certain aspects, the inflammatory disorder of the eye is pain and inflammation associated with ocular surgery, and in particular may be associated with cataract surgery. In other aspects, the difluprednate is present at a concentration of 0.04% weight by volume. In still other aspects, the method results in a pain score of 0 on the visual analog scale at day 15 post ocular surgery, where the pain score on visual analog scale may be evaluated based on eye pain and discomfort scoring from 0 to 100.

Another aspect of the present disclosure relates to an aqueous solution comprising difluprednate as the sole active ingredient at a concentration of 0.02% to 0.04% weight by volume in an aqueous vehicle for use in the reduction of inflammation in the anterior chamber of the eye of a subject that has undergone ocular surgery, wherein the solution is free of oil, wherein the solution is administered twice-a-day, wherein the subject has not received rescue medication, and wherein after the treatment the subject has an anterior chamber cell (ACC) grade of 0 and a pain score of 0 on the visual analog scale (VAS) post ocular surgery following 15 days of administration. In certain aspects, the inflammatory disorder of the eye is pain and inflammation associated with ocular surgery, and in particular may be associated with cataract surgery.

Yet another aspect of the present disclosure relates to an aqueous solution comprising difluprednate as the sole active ingredient at a concentration of 0.02% to 0.04% weight by volume in an aqueous vehicle for use in achieving an anterior chamber cell (ACC) grade of 0 in a subject in need thereof, wherein the solution is free of oil, wherein the solution is administered twice-a-day for 7-21 days, and wherein the subject has not received rescue medication. In certain aspects, the inflammatory disorder of the eye is pain and inflammation associated with ocular surgery, and in particular may be associated with cataract surgery.

Another aspect of the present disclosure relates to an aqueous solution comprising difluprednate as the sole active ingredient at a concentration of 0.02% to 0.04% weight by volume in an aqueous vehicle for use in achieving a pain score of 0 on the visual analog scale (VAS) in a subject in need thereof, wherein the solution is free of oil, wherein the solution is administered twice-a-day for 7-21 days, and wherein the subject has not received rescue medication. In certain aspects, the inflammatory disorder of the eye is pain and inflammation associated with ocular surgery, and in particular may be associated with cataract surgery.

Yet another aspect, the present disclosure relates to an aqueous solution comprising difluprednate as the sole active ingredient at a concentration of 0.02% to 0.04% w/v in an aqueous vehicle for use in the treatment of inflammatory disorder of eye, wherein the solution is free of oil and wherein the solution is administered twice-a-day for 14 days, wherein the patient has not received rescue medication, and after the treatment the patient has an anterior chamber cell grade of 0. In certain aspects, the inflammatory disorder of the eye is pain and inflammation associated with ocular surgery, and in particular may be associated with cataract surgery. In other aspects, the difluprednate is present at a concentration of 0.04% weight by volume. In still other aspects, the method results in a pain score of 0 on the visual analog scale at day 15 post ocular surgery, where the pain score on visual analog scale may be evaluated based on eye pain and discomfort scoring from 0 to 100.

In one embodiment, the efficacy of treatment was measured by biomicroscopic measurement of anterior chamber cells (ACCs) and measurement of the subject's pain level at each visit using a visual analog scale (VAS).

In some embodiments, the present disclosure provides a method of treating adverse effects of an inflammatory disorder in a population of subjects. As the skilled artisan may appreciate, in some embodiments the effect of the methods described herein on a single subject may vary. Applicants have found that the methods described herein, when used on a population of subjects, provide a measurable, consistent reduction of adverse effects associated with an inflammatory disorder. The disclosure provides a method of reducing an adverse effect associated with an inflammatory disorder of eye in a population of subjects in need thereof, said method comprising administering to the eyes of the population of subject an aqueous solution comprising difluprednate as the sole active ingredient, wherein the aqueous solution comprises 0.02% to 0.04% w/v difluprednate and an aqueous vehicle, wherein the aqueous solution is free of oil and wherein the aqueous solution is administered twice-a-day for 7 to 21 days to the population of subjects.

In some embodiments, the disclosed methods provided herein are for the reduction of adverse effects in a subject, e.g., a human subject, e.g., a female human subject or a male human subject. In some embodiments, the subject is an infant to 12 years old subject. In some embodiments, the subject is a 13 year old to 20 year old subject. In some embodiments, the subject is greater than 20 years old, e.g., 20-30 years old, 30-40 years old, 40-50 years old, or greater than 50 years old. In some embodiments, the population of subjects is a population of human subjects.

The aqueous solutions describe herein may be administered twice-a-day for greater than 7 days, greater than 10 days, greater than 12 days, greater than 14 days, or greater than 21 days. In some embodiments, the aqueous solution may be administered twice-a-day until the adverse effects are alleviated. In some embodiments, the aqueous solution is administered 12 to 16 days, or for only 14 days. The disclosure demonstrates that administration twice-a-day for as few as 14 days can provide a suitable reduction in adverse effects as described herein. Thus, in some embodiments, the aqueous solution is administered twice-a-day for 14 days.

Various forms of administration are known on the art. The disclosure provides that the current methods described herein result in the need for fewer administrations of the aqueous solution, e.g., less frequent daily administration and reduced time of administration. In some embodiments the administering is by topical application to the eye by eye drops. In some embodiments, the administration is by injection into the eye.

Anterior chamber cells (ACC): Inflammation in the anterior segment of the eye causes breakdown of the blood—aqueous barrier and results in an increase in the number of cells and the protein concentration of the aqueous humor. Examination of the aqueous humor by slit-lamp biomicroscopy is the primary method used to evaluate the severity of anterior segment inflammation.

A slit-lamp biomicroscope was used at ×16 magnification with a 1×1-mm oblique high-intensity beam. The slit illumination was turned up to its highest intensity at all available controls for illumination. The central cornea in the papillary axis was focused and the illumination was kept on the anterior aqueous and the white cells were counted only at the plane of focus. Next, the focus was moved to the central cornea and refocused in the anterior aqueous humor to determine a second count. The 2 cell counts were summed and divided by 2 to determine an average final ACC count. This final cell count was converted to a grade according to the description below. If the averaged count falls between 2 grades, the higher grade should be selected (e.g., if the 2 counts are 10 and 11, the average of 10.5 would fall into Grade 2).

Anterior Chamber Cells

| Grade | Cell Count |
|---|---|
| 0 | 0 |
| 1 | 1-10 |
| 2 | 11-20 |
| 3 | 21-50 |
| 4 | >50 |

Rescue medication: Clinical trials in specific indications require the administration of rescue medication in case a subject does not sufficiently respond to investigational treatment. But the application of additional treatment on an as needed basis causes problems to the analysis and interpretation of the results of these studies since the effect of the investigational treatment can be confounded by the additional medication. But still it is necessary to provide subjects the opportunity to receive an established medication, either alone or in combination with study treatment, in case of insufficient response. This additional treatment is called rescue medication.
Pain score: It was measured by the Visual Analogue Scale (VAS) which consists of a straight line with the endpoints defining extreme limits such as ‘Absence of Pain” and ‘pain as extreme” as it could be. Subjects were asked to rate the feeling of the symptom in their eye from absent to extreme by moving a slide on the side of the scale to align with images of descriptive faces. The scale was turned over to the other side and the associated measurement were recorded. The scale used a 100 mm (10 cm) line (0=absent; 100=maximum).

It was surprisingly found that the anti-inflammatory effect achieved by twice-a-day administration of an aqueous solution having difluprednate as the sole active ingredient at a concentration of 0.03% and 0.04% weight by volume was equivalent to the anti-inflammatory action obtained by four-times-a-day administration of an emulsion formulation of the prior art, available under the trade name of Durezol® and having difluprednate at a concentration of 0.05% w/v. This is indeed surprisingly and unexpected because the prior art composition includes a significant amount of oil, moreover it contains weight by volume of difluprednate and is instilled four time daily as compared to the aqueous solution of present disclosure having 0.04% w/v of difluprednate, which is administered twice daily and wherein the ophthalmic aqueous solution is free of oil. This is all the more surprising because difluprednate being hydrophobic/oil soluble is considered to remain in the oil phase and thus, expected to provide better efficacy in an emulsion type of composition compared to a composition which is aqueous based and most importantly, free of oil. The results found by the inventors are in fact contrary to the established hypothesis.

The present disclosure provides a clear aqueous solution formulation of difluprednate for use in treatment of an inflammatory disorder of the eye which can be administered twice-a-day with the added advantage that the solution form enables use of lower concentration of difluprednate as compared to the existing prior art compositions or marketed products.

The present disclosure provides a remarkable improvement in the method of treatment of an inflammatory disorder of the eye. By virtue of the clear nature of the aqueous solution being free of any suspended particles, reduced frequency of administration and potential for use of reduced drug concentration, thus enhanced ocular bioavailability, the method not only provides an enhanced subject compliance, but also avoids the untoward side effects such as blurred vision, irritation, foreign body sensation, etc., upon instillation.

In one embodiment, the ophthalmic aqueous solution of difluprednate is useful in the treatment of pain and inflammation associated with ocular surgery by twice-a-day instillation into the effected eye of the subject. In another embodiment, the ophthalmic aqueous solution of the present disclosure is useful in the treatment of pain and inflammation associated with cataract surgery by twice-a-day instillation into the effected eye of the subject.

The inflammatory disorders of the eye that can be treated by administering ophthalmic aqueous solution according to the present disclosure include, but are not limited to, pain and inflammation associated with ocular surgery, uveitis, acute anterior uveitis, endogenous anterior uveitis, chronic uveitis, inflammation associated with ocular allergies, steroid responsive inflammatory condition of the palpebral and bulber conjunctiva, cornea and anterior segment of the globe such as allergic conjunctivitis, acne rosacea, superficial punctuate keratitis, and herpes zoster keratitis. In some embodiments, the ophthalmic aqueous solution according to the present disclosure is useful in the treatment of various forms of uveitis, such as iritis or anterior uveitis, iridocyclitis and choroiditis or chorioretinitis also known as posterior uveitis, acute anterior uveitis, and chronic uveitis.

In one embodiment, the present disclosure provides a method of treating pain and inflammation associated with ocular surgery by twice-a-day instillation into the effected eye of the subject, an ophthalmic aqueous solution comprising difluprednate at a concentration of about 0.02% to 0.04% weight by volume, wherein the solution is free of oil, and wherein the subject has not received rescue medication and after the treatment the subject had an anterior chamber cell (ACC) grade of 0 and pain score of 0 on the visual analog scale (VAS) post ocular surgery.

In one embodiment, the present disclosure provides a method of treating an inflammatory disorder of the eye, said method comprising administering into the eye of a person in need thereof, an aqueous solution comprising difluprednate as the sole active ingredient at a concentration of about 0.02% to 0.04% weight by volume, wherein the solution is free of oil and wherein the solution is administered twice-a-day, further wherein the aqueous solution comprises a crystal growth inhibitor and pharmaceutically acceptable amounts of a solubilizer comprising a mixture of a quaternary ammonium compound, and polyethoxylated castor oil. The crystal growth inhibitor is polyvinyl pyrrolidone or its derivative, the inflammatory disorder of the eye is pain and inflammation associated with ocular surgery, wherein the subject has not received rescue medication and after the treatment the subject had an anterior chamber cell (ACC) grade of 0 and pain score of 0 on the visual analog scale (VAS) post ocular surgery.

The ophthalmic aqueous solution of the present disclosure is capable of enhancing the ocular bioavailability of difluprednate and thus decreasing its frequency of administration. Whereas DUREZOL® the marketed emulsion product of difluprednate is instilled four times a day, the aqueous solution of the present disclosure requires only twice daily instillation to achieve the desired therapeutic effect.

In one embodiment, the present disclosure thus provides a method of enhancing the ocular bioavailability of difluprednate, said method comprising twice-daily instillation into the eye of a person in need thereof, an ophthalmic aqueous solution of difluprednate.

In one embodiment, the present disclosure provides a method of enhancing the ocular bioavailability of difluprednate, said method comprising twice-a-day instillation into the eye of a person in need thereof, an ophthalmic solution comprising therapeutically effective concentration of difluprednate, a crystal growth inhibitor and pharmaceutically acceptable amounts of a solubilizer comprising a mixture of a quaternary ammonium compound, and polyethoxylated castor oil, in an aqueous vehicle.

The ophthalmic aqueous solutions of the present disclosure enhance the ocular bioavailability of difluprednate and reduce the frequency of administration to twice-a-day administration, for achieving the desired anti-inflammatory effect, as against the repeated four times a day administration required for a marketed emulsion product (DUREZOL®).

### EXAMPLES

While the present disclosure is disclosed generally above, additional aspects are further discussed and illustrated with reference to the examples below. However, the examples are presented merely to illustrate the disclosure and should not be considered as limitations thereto.

### Examples 1-4

The examples describe ophthalmic aqueous solutions of difluprednate according to the present disclosure.

TABLE 1
Ophthalmic aqueous solutions of difluprednate

| | Quantity (% w/v) | | | |
|---|---|---|---|---|
| Ingredients | Example 1 | Example 2 | Example 3 | Example 4 |
| Difluprednate | 0.03 | 0.04 | 0.04 | 0.04 |
| Benzalkonium chloride | 0.02 | 0.02 | 0.02 | 0.025 |
| N-lauroyl sarcosine | 0.02 | 0.02 | 0.02 | 0.03 |
| Polyoxyl 35 castor oil | 4.0 | 4.0 | 4.0 | 5.00 |
| (Cremophor ® EL) | | | | |
| Polyvinyl alcohol | 1.4 | 1.4 | 1.4 | 1.40 |
| Carboxy methyl | — | — | 0.3 | — |
| cellulose | | | | |
| Glycerine | 2.0 | 2.0 | 2.0 | 1.60 |
| Boric acid | 0.50 | 0.5 | 0.5 | 0.6 |
| Poly hexa methylene | — | — | — | 0.005 |
| biguanide | | | | |
| Disodium edetate | 0.05 | 0.05 | 0.05 | 0.05 |
| Sodium acetate | 0.025 | 0.025 | 0.025 | 0.01 |
| Acetic acid | — | — | — | 0.005 |
| Water for Injection | q.s to 100 | q.s to 100 | q.s to 100 | q.s to 100 |

The required quantity of benzalkonium chloride and difluprednate was taken in a container and polyoxyl 35 castor oil (Cremophor®) was added. Difluprednate was solubilized in the above mixture by intermittent sonication and vortexing to form drug reconcentrate.

The required quantity of polyvinyl alcohol was dissolved in water for injection at The other ingredients, i.e. boric acid, N-lauryl sarcosine, glycerine, disodium edetate, sodium acetate, and acetic acid, were separately dissolved in a portion of water for injection.

This phase was added to polyvinyl alcohol solution with stirring. Drug preconcentrate was added to the above mixture and dissolved by mixing with a magnetic stirrer to get a solution (i). In case of example 4, an additional step was followed wherein required quantity of polyhexa methylene biguanide was added to the solution (i). In case of example 3, an additional step was followed wherein carboxymethyl cellulose pre-mixed with a portion of water was added to the solution (i) obtained above, with stirring.

Subsequently volume make up was carried out using water for injection. The solution was filtered with 0.2 micron filter. The resulting ophthalmic aqueous solutions of difluprednate (Examples 1-4) had a pH of about 5.0-6.0, osmolality of about 300 mOsm/kg and percentage transmittance of about 99%. The viscosity of ophthalmic aqueous solution of Example 1, 2, and 4 was about 4-5 cps, and the viscosity of ophthalmic aqueous solution of Example 3 was about 20 cps.

The physicochemical stability of the formulations was tested upon storage at room temperature (25/40% relative humidity) and at 2-8° C. for 6 months. It was found that the solutions of Examples 1-4 remained clear and free from particles, crystals or precipitate upon storage. The percentage transmission was greater than 95% upon storage. The assay of difluprednate remained in the range of 95%-105% w/v, the known and unknown impurities did not increased substantially upon storage and the content of impurities remained below the desired specified limit.

### Comparative Examples 1-3

This example illustrates comparative non-working examples 1, 2 and 3 (which are devoid of a solubilizer as per the disclosure) and their comparison with working Examples and 6 as per the disclosure (which contains a solubilizer having a mixture of a quarternary ammonium compound and polyethoxylated castor oil). These examples illustrate the surprising effect of use of a mixture of a quarternary ammonium compound and a polyethoxylated castor oil (Cremophor®) whose combination acts as an efficient solubilizer of difluprednate in an aqueous vehicle.

TABLE 2
Effect of mixture of solubilizer versus single solubilizer versus absence of solubilizer:

| | Comparative Examples | | | Example of the present | | |
|---|---|---|---|---|---|---|
| | 1 | 2 | 3 | 5 | 6 (a) | 6 (b) |
| Ingredients | Quantity % w/v | | | Quantity % w/v | | |
| Difluprednate | 0.04 | 0.04 | 0.04 | 0.04 | 0.04 | 0.04 |
| Benzalkonium Chloride | — | 0.02 | — | 0.02 | 0.02 | 0.02 |
| N-Lauryl Sarcosine | 0.02 | 0.02 | 0.02 | 0.02 | — | 0.02 |
| Polyethoxylated castor | — | — | 4.00 | 4.00 | 4.00 | 4.00 |
| oil (Cremophor ® EL) | | | | | | |
| Polyvinyl Alcohol | 1.40 | 1.40 | 1.40 | 1.40 | 1.40 | — |
| Polyvinyl Alcohol-PEG graft | — | | | | | 3.00 |
| co-polymer | | | | | | |
| Glycerol | 2.20 | 2.20 | 2.20 | 2.20 | 2.20 | 2.20 |

| Water for Injection | q.s to 100 | | |
|---|---|---|---|
| Observation upon storage at room | Immediate | Precipitation | No precipitation till |
| temperature | Precipitation- | within | 6 months (−) |
| Time Point at which precipitation | clear solution | 2 day | |
| observed (+); | not formed | (+) | |
| No precipitation (−) | (+++) | | |

Benzalkonium chloride is a widely used component as a preservative in ophthalmic formulations. However, the inventors found that in the present invention benzalkonium chloride plays an important role for the solubilisation and stabilization of the difluprednate molecule in aqueous system when used along with a non-ionic surfactant like Cremophor®.

The inventors found that a combination of a quarternary ammonium compound, such as benzalkonium chloride, and a non-ionic surfactant, such as polyethoxylated castor oil (Cremophor®), solubilizes the hydrophobic drug-difluprednate in an aqueous vehicle and leads to formation of a storage stable clear aqueous solution, (working examples 5 and 6), which solution remains clear and there occurs no precipitation or crystallization of difluprednate upon long term storage (at least 6 months) at room temperature.

The inventors observed that use of a quarternary ammonium compound along with a non-ionic surfactant, Cremophor®, is necessarily required for the solubilization of difluprednate. It was found that the quarternary ammonium compound along with Cremophor® plays an important role for the solubilisation and stabilization of difluprednate molecule in aqueous system. Mainly, when the two components are present together, the drug is solubilized in an aqueous vehicle.

However, in absence of either or both of a quarternary ammonium compound (like benzalkonium chloride) or polyethoxylated castor oil (Cremophor C)) proper solubilisation of difluprednate in aqueous solution does not take place and the resulting formulations were unstable such that difluprednate gets crystallized out or precipitated out (comparative examples 1-3).

### Comparative Examples 4-10

These comparative examples tests the effect of various crystal growth inhibitors on solubilisation and stabilization of difluprednate in an aqueous solutions:

Aqueous solutions of difluprednate were prepared by substituting polyvinyl alcohol with other crystal growth inhibitors like hydroxypropyl methylcellulose, hydroxyl ethyl cellulose, hydroxyethyl starch, polyvinylpyrrolidone, carboxyvinyl polymer and the like. The physical stability of the solutions was studied upon storage at room temperature. The details of the quantitative formulations tested along with stability study results are presented below in Table 3:

TABLE 3
Effect of crystal growth inhibitors

| Comparative Examples | | | | | | |
|---|---|---|---|---|---|---|
| 4 | 5 | 6 | 7 | 8 | 9 | 10 |

| Category | Ingredients | Amount (% w/v) | | | | | |
|---|---|---|---|---|---|---|---|
| Anti- | Difluprednate | 0.04 | | | | | |
| inflammatory | | | | | | | |
| Solubilizer | Benzalkonium | 0.02 | | | | | |
| | Chloride | | | | | | |
| | Polyethoxylated | 4.00 | | | | | |
| | castor oil | | | | | | |
| | (Cremophor ® | | | | | | |
| | EL) | | | | | | |
| Preservative | N-Lauryl | 0.02 | | | | | |
| | Sarcosine | | | | | | |
| Co-solvent | Glycerol | 2.20 | | | | | |
| Excipients | Hydroxypropyl | 0.2 | — | | | | |
| tried for | methyl | | | | | | |
| their | cellulose | | | | | | |
| precipitation/ | Polyvinyl- | — | 1.0 | — | | | |
| crystal | pyrrolidone | | | | | | |
| growth | (Povidone ® | | | | | | |
| inhibition | K-90) | | | | | | |
| effect | Carboxyvinyl | — | | 0.1 | — | | |
| | polymer | | | | | | |
| | (Carbopol ®934) | | | | | | |
| | Hydroxyethyl | — | | | 1 | — | |
| | starch | | | | | | |
| | Guar gum | | | | | 0.2 | — |

| | | | |
|---|---|---|---|
| Polyoxyethylene- | — | 1 | — |

polyoxypropylene
co-polymer
(Pluronic F-68)

| | | |
|---|---|---|
| Vehicle | Water for | q.s. to 100 |
| | Injection | |

| | | | |
|---|---|---|---|
| Observation upon storage | 5 | within | 12 |
| at room temperature | week | 24 hours | days |
| Time Point at which | (+) | | (+) |
| precipitation observed (+); | | (+) | |

The inventors of the present disclosure surprisingly discovered that only a particular polymer that is, polyvinyl alcohol or its derivatives, works as efficient crystal growth inhibitor, which maintains proper solubilization of difluprednate in aqueous solution, and prevents crystallization or precipitation of drug upon long term storage at room temperature. (Working examples 1-6 of the present disclosure). On the other hand, when other polymers or surfactants other than polyvinyl alcohol were used, (comparative examples 4-9), it was found that there occurred precipitation or crystallization of drug, at varying time points as given in Table 3.

### Example 7

Preservative Efficacy Testing: The formulation of Example 4 was tested for efficacy of antimicrobial preservation, as per the preservative efficacy test specified in the European Pharmacopoeia, 7.0 Edition, section 5.3.1, Page 505-506, which is herein incorporated by reference in its entirety.

The results of Preservative Efficacy Testing for bacteria as per acceptance criteria's A and B specified in the European Pharmacopoeia are presented below in Table 4:

TABLE 4
Results for Preservative Efficacy Testing:
PET Results - European Pharmacopoeia Criteria-A

| | | |
|---|---|---|
| | Log | |
| | reduction for | |
| Bacteria | compliance | Observation |

Bacteria Log reduction At 6 Hr

| | | |
|---|---|---|
| P. Aeruginosa | NLT 2.0 | 5.16 |
| S. Aureus | NLT 2.0 | 5.05 |

Bacteria Log reduction At 24 Hr

| | | |
|---|---|---|
| P. Aeruginosa | NLT 3.0 | 5.16 |
| S. Aureus | NLT 3.0 | 5.05 |

Bacteria Log reduction At 28 Days

| | | |
|---|---|---|
| P. Aeruginosa | No recovery | 5.16 |
| S. Aureus | No recovery | 5.05 |
| Result as per | | Complies |
| bacteria log | | |
| reduction | | |

PET Results - European Pharmacopoeia Criteria-B

| | |
|---|---|
| | Log |
| | reduction for |
| Bacteria | compliance |

Bacteria Log reduction At 24 Hr

| | | |
|---|---|---|
| P. Aeruginosa | NLT 1.0 | 5.16 |
| S. Aureus | NLT 1.0 | 5.05 |

Bacteria Log reduction At 7 Day

| | | |
|---|---|---|
| P. Aeruginosa | NLT 3.0 | 5.16 |
| S. Aureus | NLT 3.0 | 5.05 |

Bacteria Log reduction At 28 Days

| | | |
|---|---|---|
| P. Aeruginosa | No increase | 5.16 |
| S. Aureus | No increase | 5.05 |
| Result as per | | Complies |
| bacteria log | | |
| reduction | | |

It was observed that the aqueous solution of the present disclosure (example 4) complies with the specifications as per acceptance criteria's A and B of efficacy of antimicrobial preservation test defined in European Pharmacopoeia. i.e. required log reduction for the bacteria at 6 h, 24 h and 7 day as per criteria A and 24 h, 7 day and 28 day as per criteria B was achieved. The test of efficacy of antimicrobial preservation was also performed on another batch of aqueous solution which did not contain a biguanide and it was observed that the efficacy of antimicrobial preservation was inferior to that observed in the batch having a biguanide, inclusion of a biguanide, like polyhexamethylene biguanide, helps in enhancing the anti-microbial efficacy of the aqueous solution.

### Example 8

Effect of Different Preservatives on Antimicrobial Efficacy

These comparative examples test the antimicrobial efficacy of different preservatives in difluprednate aqueous solutions. These examples illustrate the surprising effect of use of a preservative mixture of benzalkonium chloride, N-lauroyl sarcosine and boric acid. To establish the inclusion of these ingredients, various compositions were prepared with and without the preservatives and preservative mixtures and tested for Antimicrobial Efficacy Test.

TABLE 5
Results for Antimicrobial Efficacy Testing (AET):

| | Amount (% w/v) | | | | |
|---|---|---|---|---|---|
| | Example | Example | Example | Example | Example |
| Ingredients | 1 | 2 | 3 | 4 | 5 |
| Difluprednate | 0.04 | 0.04 | 0.04 | 0.04 | 0.02 |
| Benzalkonium chloride | — | — | 0.04 | 0.04 | 0.02 |
| N-lauroyl sarcosine | — | 0.02 | — | 0.02 | 0.02 |
| Polyoxyl 35 castor oil | 4.00 | 4.00 | 4.00 | 4.00 | 4.00 |
| (Cremophor ®) | | | | | |
| Polyvinyl Alcohol | 1.40 | 1.40 | 1.40 | 1.40 | 1.40 |
| Glycerol | 2.20 | 2.20 | 2.20 | 2.20 | 2.20 |
| Boric Acid | 0.25 | 0.25 | 0.25 | — | 1.00 |
| Carboxy methyl cellulose | — | — | — | — | 0.50 |
| Water for Injection | q.s. to | q.s to | q.s. to | q.s. to | q.s. to |
| | 100 | 100 | 100 | 100 | 100 |

It was observed that the presence of boric acid in the aqueous solutions of the compositions helped to comply with the specification as per acceptance criteria of Antimicrobial Efficacy test defined in United States Pharmacopoeia. When N-lauroyl sarcosine was used alone as a preservative (Example 2), the batch did not comply with the acceptance criteria of Antimicrobial Efficacy test as defined in United States Pharmacopoeia and criteria's A and B as defined in European Pharmacopoeia. When boric acid and benzalkonium chloride both were present in the formulation (Example 3 and Example 5), the batches complied with the antimicrobial efficacy testing as per United States Pharmacopoeia. Inclusion of N-lauroyl sarcosine (Example 5) helped the formulation to comply with the Criteria B of antibacterial activity as per the European Pharmacopoeia.

Table 6 below shows the results of antimicrobial efficacy testing of batches containing a mixture of preservatives comprising benzalkonium chloride, N-lauroyl sarcosine, boric acid and polyhexamethylene biguanide. In addition, glacial acetic acid and EDTA were added to meet the desired physicochemical characteristics of the drug product. Also, the quantities of benzalkonium chloride, boric acid, sodium acetate and glycerin were optimized to meet target physicochemical characteristics.

TABLE 6
Results for Antimicrobial Efficacy Testing (AET):

| | Quantity (% w/v) | |
|---|---|---|
| Ingredients | Example 1 | Example 2 |
| Difluprednate | 0.04 | 0.04 |
| Benzalkonium chloride | 0.05 | 0.05 |
| N-lauroyl sarcosine | 0.03 | 0.03 |
| Polyoxyl 35 castor oil (Cremophor ® EL) | 5.00 | 5.00 |
| Polyvinyl alcohol | 1.40 | 1.40 |
| Glycerine | 1.60 | 1.50 |
| Boric acid | 0.60 | 0.60 |
| Poly hexa methylene biguanide | 0.005 | 0.005 |
| Disodium edetate | 0.05 | 0.05 |
| Sodium acetate | 0.025 | 0.01 |
| Acetic acid | — | 0.005 |
| Water for Injection | q.s to 100 | q.s to 100 |

It was surprisingly found that when a mixture of preservatives comprising benzalkonium chloride, N-lauroyl sarcosine, boric acid and polyhexamethylene biguanide was used, both batches (Examples 1 and 2) passed the Antimicrobial Efficacy Testing criteria as per United States Pharmacopoeia and Criteria B of Antimicrobial Efficacy Test as defined in European Pharmacopoeia. Thus, use of a mixture of preservatives helps in enhancing the antimicrobial efficacy of the aqueous solution.

### Example 9

Animal efficacy study in bovine serum albumin induced chronic uveitis model—Efficacy of difluprednate ophthalmic aqueous solution of the present disclosure were tested in bovine serum albumin induced chronic uveitis model in NZW rabbits and a comparison was made with marketed Durezol® formulation. Various formulations which were tested include:
Placebo, i.e. formulation vehicle similar to Example 1 but not having difluprednate.
Difluprednate ophthalmic aqueous solution of Example 1 having 0.03% w/v of difluprednate.
Difluprednate ophthalmic aqueous solution of Example 2 having 0.04% w/v of difluprednate.
Reference Item, Durezol®—Difluprednate (0.05% w/v) ophthalmic emulsion formulation by Alcon.

The study was performed in NZW rabbits, weighing 3 to 5 kg. The animals were divided into 7 groups, with 5 animals in each group. The 7 groups included—
Group 1—Normal control group
Group 2—Water for injection (WFI) group
Group 3—BSA or bovine serum albumin (disease control) group
Group 4—Placebo group
Group 5—Example 1 group
Group 6—Example 2 group
Group 7—Reference item (Durezol®) group

On day 0, all the animals except normal control group were anesthetized with Ketamine and Xylazine by intramuscular route and one drop of lignocaine was applied in each eye for topical anesthesia. On day 0, in group 3 to 8, 200 μL of BSA(5%) was injected intravitreally in both eyes and on day 7 animals were challenged with 2.5 mL of BSA (2%) intravenous injection in marginal ear vein. On day 0, in WFI control group (group 2), 200 μL of sterile WFI was injected intravitreally in the same way mentioned above and on day 7, 2.5 mL of WFI was administered intravenously by marginal ear vein.

The placebo, the Example 1 solution, Example 2 solution and the reference item were administered after 1 hour of intravenous challenge with BSA on the day 7 to respective group of animals and the administration was subsequently carried out till day 27 of the study. 50 μL of placebo, Example 1 solution and Example 2 solution were instilled topically two times at 12 hours interval to both eye of respective group of animals using micropipette from day 7 to day 27. 50 μL of reference item was instilled four times at 4 hour interval to the both eye of respective group of animals using micropipette from day 7 to day 27. The normal control, WFI and BSA (disease control) animals remain untreated. On day 14, 21 and 28, each animal was anesthetized using ketamine and Xylazine intramuscular injection. The eyes were examined for clinical grading using Zeiss slit lamp. The clinical evaluation of uveitis included evaluation of Total Clinical Score (on a 0 to 10 point basis as given in Table 5) on Day 14, 21 and 28. The clinical evaluation of uveitis further included evaluation of Total Cell Count and Total Protein in Aqueous Humor on Day 28.

TABLE 7
Clinical signs and grade of Uveitis

| Clinical Signs | Grade of Uveitis (Score) |
|---|---|
| Iris hyperemia | |
| Absent | 0 |
| Mild | 1 |
| Moderate | 2 |
| Severe | 3 |
| Dilation of the iris and conjunctival vessels | |
| Absent | 0 |
| Mild | 1 |
| Moderate | 2 |
| Cell Exudate in anterior chamber | |
| Absent | 0 |
| Mild | 1 |
| Moderate | 2 |
| Severe | 3 |
| Presence of fibrinoid exudation in the | |
| pupillary area, with intense flare in the | |
| anterior chamber | |
| Absent | 0 |
| Mild | 1 |
| Moderate | 2 |
| Total Maximum Clinical Score | 10 |

Observations: The observations of the Total Clinical Score (0-10) on day 14, 21 and 28; Total Cell Count and Total Protein in Aqueous humor on day 28, are presented below in Table 8:

TABLE 8
Observations

| | | | | | | | TLC | | | |
|---|---|---|---|---|---|---|---|---|---|---|
| | | | | | | | (10⁴)/ml of | | Total | |
| | | | | | | | aqueous | | Protein | |
| | Total Clinical Score (0-10) | | | | | | humour | | (pg/ml) | |
| | Day 14 | | Day 21 | | Day 28 | | Day 28 | | Day 28 | |
| Groups | Mean | SD | Mean | SD | Mean | SD | Mean | SEM | Mean | SD |
| Normal Control | 1.00 | 0.9 | 0.80 | 1.0 | 1.40 | 1.1 | 0.00 | 0.00 | 1.09 | 1.09 |
| WFI Control | 1.20 | 1.2 | 1.20 | 0.9 | 1.40 | 0.5 | 0.00 | 0.00 | 0.95 | 0.76 |
| BSA (Disease Control) | 5.30 | 2.8 | 7.60 | 1.1 | 7.80 | 0.8 | 10.75 | 3.65 | 15.91 | 6.26 |
| | | *** | | *** | | *** | | *** | | *** |
| Placebo | 6.30 | 0.9 | 7.70 | 1.6 | 8.00 | 0.9 | 12.70 | 4.74 | 13.53 | 6.11 |
| | | ns | | ns | | ns | | ns | | ns |
| Example 1 | 4.20 | 0.8 | 2.90 | 2.3 | 3.40 | 2.6 | 2.85 | 2.77 | 3.24 | 2.91 |
| group | | $ | | $$$ | | $$$ | | $$$ | | $$$ |
| Example 2 | 5.10 | 1.3 | 3.50 | 3.3 | 3.30 | 2.6 | 2.15 | 0.97 | 4.09 | 3.57 |
| group | | ns | | $$$ | | $$$ | | $$$ | | $$$ |
| Reference | 4.70 | 2.5 | 3.60 | 2.2 | 3.30 | 2.0 | 3.05 | 1.21 | 4.29 | 4.80 |
| Item-Durezol ® | | ns | | @@@ | | @@@ | | @@@ | | @@@ |
| group | | | | | | | | | | |

Total clinical score Data were analyzed using Two way ANOVA followed by Bornferroni test WFI vs BSA (Disease control); *** = p < 0.001, BSA (Disease control) vs Placebo; ns = non-significant, Placebo vs example 1, example 2; ns = non-significant, $ = p < 0.05, $$ = p < 0.01, $$$ = p < 0.001, BSA (Disease control) vs Reference item Durezol ®; ns = non-significant, @@@ = p < 0.001

Total cell count and Total protein: Data were analyzed using One way ANOVA followed by Bornferroni test WFI vs BSA (Disease control);***=p<0.001, BSA (Disease control) vs Placebo; ns=non significant, Placebo vs example 1, example 2; ns=non significant;$$$=p<0.001, BSA (Disease control) vs Reference item Durezol®; @ @ @=p<0.001

It was observed that the difluprednate ophthalmic aqueous solution of the present disclosure having 0.03% w/v-0.04% w/v difluprednate (Examples 1 and 2) when instilled twice-a-day into the eye effected by chronic uveitis showed a significant inhibition in total clinical score, total cell count and total protein as compared to Placebo. Particularly, in the case of the Example 1 group, wherein the aqueous solution has 0.03% w/v of difluprednate, the mean clinical score reduced significantly from 7.7 (placebo) to 2.9 at day 21 and from 8.0 (placebo) to 3.4 at day 28. Similarly, in case of the Example 2 group, wherein the formulation has 0.04% w/v of difluprednate, the mean clinical score reduced significantly from 7.7 (placebo) to 3.5 at day 21 and from 8.0 (placebo) to 3.3 at day 28.

The total clinical score, total cell count and total protein levels were also significantly attenuated by reference item Durezol® as compared to the BSA (disease control) group.

The clinical score values, the total cell count and total protein content in aqueous humor observed at day 14, 21 and 28 by twice-a-day administration of low concentration (0.03% and 0.04%) difluprednate ophthalmic aqueous solution of the present disclosure was equivalent or better than that observed upon four times a day administration of higher concentration 0.05% w/v emulsion formulation Durezol® (marketed reference item).

### Example 10

Animal efficacy study in lipopolysaccharide (LPS) induced acute uveitis model —Efficacy of difluprednate ophthalmic aqueous solutions of the present disclosure were tested in lipopolysaccharide (LPS) (an endotoxin) induced acute uveitis in female NZW rabbits and a comparison was made with a marketed Durezol® formulation. Various formulations which were tested include:
Placebo: Formulation vehicle similar to Example 1 but not having difluprednate.
Difluprednate ophthalmic aqueous solution of Example 1 having 0.03% w/v of difluprednate.
Difluprednate ophthalmic aqueous solution of Example 2 having 0.04% w/v of difluprednate.
Durezol® Difluprednate (0.05% w/v) ophthalmic emulsion formulation by Alcon.

The study was performed in NZW rabbits, weighing 3 to 5 kg. The animals were divided into 7 groups, with 5 animals in each group. The 7 groups included —
Group 1—Normal control group
Group 2—Phosphate buffer saline (PBS) group
Group 3—Lipopolysaccharide (LPS disease control) group
Group 4—Placebo group
Group 5—example 1 group
Group 6—example 2 group
Group 7—Reference item (Durezol®) group.

On day 0, all the animals except normal control group were anesthetized with Ketamine and Xylazine by intramuscular route and one drop of lignocaine was applied in each eye for topical anaesthesia. On day 0, in PBS control group, 20 μL of sterile phosphate buffer saline pH-7.4 was injected intravitreally in both eyes. In groups 3 to 8, 20 μL of LPS (100 ng) was injected intravitreally in both eyes The placebo, the Examples 1 and 2 solutions and reference item were administered 1 hour after LPS injection, wherein 50 μL of placebo and Examples 1 and 2 solutions were instilled topically two times at 12 hours interval to both eye of respective group of animals using micropipette, while 50 μL of reference item was instilled four times at 4 hour interval to both eyes of respective group of animals using micropipette. The normal control, PBS and LPS (disease control) animals remain untreated. After 24 hours of LPS injection, each animal was anesthetized using ketamine and Xylazine intramuscular injection. The eyes were examined for clinical grading using Zeiss slit lamp.

The clinical evaluation of uveitis included evaluation of Total Clinical Score (on a 0 to 5 point basis as given in Table 7 below) on Day 1. The clinical evaluation of uveitis further included evaluation of Total Cell Count and Total Protein in aqueous humor on Day 1.

Aqueous humor was collected from each animal using 30 gauge needle attached with appropriate syringe after clinical scoring. Aqueous humor samples were stored at 2-8° C. till analysis. The total cell count and total protein of each animal were calculated.

TABLE 9
Clinical signs and grade of Uveitis

| Clinical Signs | Grade of Uveitis (Score) |
|---|---|
| Iris hyperemia | |
| Absent | 0 |
| Mild | 1 |
| Moderate | 2 |
| Severe | 3 |
| Pupil | |
| Normal | 0 |
| After Miosis | 1 |
| Exudate in anterior chamber | |
| Absent | 0 |
| Present | 1 |
| Total Maximum Clinical Score | 5 |

Observations: The observations of the Total Clinical Score (0-5); Total Cell Count and Total Protein in aqueous humor on day 1, are presented below in Table 10:

TABLE 10
Observations

| | Total | | TLC | | | |
|---|---|---|---|---|---|---|
| | Clinical | | (10⁴)/ml of | | Total | |
| | Score | | aqueous | | Protein | |
| | (0-5) | | humour | | (pg/ml) | |
| Groups | Mean | SD | Mean | SEM | Mean | SD |
| Normal Control | 0.0 | 0.0 | 0.15 | 0.1 | 0.91 | 0.6 |
| PBS Control | 0.3 | 0.5 | 0.15 | 0.1 | 1.04 | 0.3 |
| LPS (Disease Control) | 3.1 | 0.7 | 231.75 | 87.4 | 21.94 | 4.7 |
| | | ### | | # | | ### |
| Placebo | 3.0 | 1.1 | 224.8 | 71.4 | 20.23 | 3.9 |
| | | ns | | Ns | | Ns |
| Example 1 | 1.0 | 0.9 | 30.2 | 12.9 | 9.65 | 8.8 |
| | | $$$ | | $ | | $ |
| Example 2 | 0.7 | 0.8 | 29.56 | 18.6 | 10.11 | 9.1 |
| | | $$$ | | $ | | $ |
| Durezol | 0.7 | 0.5 | 21.2 | 9.8 | 13.21 | 8.6 |
| | | *** | | * | | * |

Total clinical score:
PBS vs LPS (Disease control) = t-test (# = p < 0.05, ### = p < 0.001), LPS (Disease control) vs Placebo = t-test; (ns = non significant); Placebo vs example 1 and 2 = One way ANOVA followed by Dunnett's Multiple Comparison Test ($ = p < 0.05, $$ = p < 0.01, $$$ = p < 0.001),; LPS (Disease control) vs Reference Durezol = t-test (* = p < 0.05, ** = p < 0.01, *** = p < 0.001); Durezol vs example 1, example 2 = One way ANOVA followed by Dunnett's Multiple Comparison Test ($ = p < 0.05, ns = non-significant)

It was observed that the difluprednate ophthalmic aqueous solution of the present disclosure having 0.03% w/v-0.04% w/v difluprednate (Examples 1 and 2) when instilled twice-a-day into the eye effected by acute uveitis showed a significant inhibition in total clinical score, total cell count and total protein as compared to Placebo. Particularly, in case of the Example 1 group, (0.03% w/v difluprednate solution), the mean clinical score reduced significantly from 3.0 (placebo) to 1.0 at day 1. Similarly, in case of the Example 2 group, (0.04% w/v difluprednate solution), the mean clinical score reduced significantly from 3.0 (placebo) to 0.7.

The total clinical score, total cell count and total protein levels were also significantly attenuated by reference item Durezol® as compared to BSA (disease control) group. Particularly, the mean clinical score reduced from 3.0 (placebo) to 0.7 at day 1.

The clinical score values, the total cell count and total protein content in aqueous humor observed at day 1 by twice-a-day administration of low concentration (0.03% w/v and 0.04% w/v) difluprednate ophthalmic aqueous solutions of the present disclosure were equivalent or better than that observed upon four times a day administration of higher strength 0.05% w/v emulsion formulation Durezol® (marketed reference item).

### Example 11

A Randomized, double-masked, parallel group, multicentre, study was conducted to evaluate efficacy and safety of a difluprednate ophthalmic solution 0.04% twice daily compared with vehicle for the treatment of inflammation and pain associated with ocular surgery.

In this study, 357 subjects were screened. Out of 357, 325 subjects were randomized intent-to-treat (ITT) population, while 32 subjects failed the inclusion/exclusion criteria and were considered as screen failures. A total of 26 subjects were randomized but were never dosed.

Out of 299 treated subjects, at least one dose of Test product was received by 154 subjects while 145 subjects received Reference product. The modified intent-to-treat (mITT) data set included randomized subjects who underwent cataract surgery and received at least one dose of Investigational Product (IP) and had at least one post-surgery efficacy assessment (Test: 154 and Reference: 144). The Per protocol data set included all mITT subjects who completed evaluation at test of cure visit at the postoperative day 15 visit with no protocol violations that would affect treatment evaluation (Test: 123 and Reference: 83). A total of 232 subjects completed the study, while 21 subjects in Test group and 72 subjects in Reference group discontinued the study due to various reasons.

Subjects of both genders were enrolled. In the Test group, 64 (39.3%) subjects were Male while 99 (60.7%) were Female. Their average age was 68.4 yrs. In the Reference group, 68 (42.0%) subjects were Male and 94 (58.0%) were Female, with an average age of 68.6 yrs.

In this study, 240 (73.85) subjects were White, while 73 (22.5%) were Black or African American, 9 (2.8%) were Asians and 3 (90.9%) were Other. Races were equally distributed between the two treatment groups. A similar trend was seen for Ethnicity

[Hispanic or Latino, 85 (26.2%) and Not Hispanic or Latino 239 (73.5%)].

Primary Efficacy Analysis: Proportion of Subjects with an Anterior Chamber Cell (ACC) Grade of Zero (0) at Day 15

A subject with an ACC grade of 0 at the Day 15 visit or the last assessment prior to the Day 15 visit (in the event that Day 15 was not completed) was considered a responder to therapy. And if a subject had an ACC score of >0 at Day 15 or the last assessment prior to the Day 15 visit (in the event that Day 15 was not completed), that subject was considered a failure (or non-responder) in the primary efficacy endpoint analysis. Additionally, any subject receiving a rescue medication between Day 0 and the Day 15 visit (inclusive) was considered a treatment failure.

Accordingly, considering the mITT population (N=299), the Test group recorded 60.4% responders while the Reference group recorded 20.8% responders. A statistically significant result (p value<0.0001) favouring Test drug showed that the primary objective was met. A similar trend was seen in the Per Protocol (PP) population (p value<0.0001).

TABLE 11
Observations

| | mITT Population | | PP Population | |
|---|---|---|---|---|
| | Test | | Test | |
| | Product | Reference | Product | Reference |
| ACC | (N = 154) | (N = 144) | (N = 123) | (N = 83) |
| Grade^{[1][2]} | n(%) | n(%) | n(%) | n(%) |

Responders

| | | | | | | | | |
|---|---|---|---|---|---|---|---|---|
| 0 (Did not receive | 93 | (60.4) | 30 | (20.8) | 84 | (68.3) | 27 | (32.5) |
| rescue therapy) | | | | | | | | |
| Non-Responders | | | | | | | | |
| 0 (Received | 3 | (1.9) | 0 | (0.0) | 0 | (0.0) | 0 | (0.0) |
| rescue therapy) | | | | | | | | |
| 1 | 50 | (32.5) | 46 | (31.9) | 38 | (30.9) | 42 | (50.6) |
| 2 | 1 | (0.6) | 12 | (8.3) | 1 | (0.8) | 13 | (15.7) |
| 3 | 0 | (0.0) | 2 | (1.4) | 0 | (0.0) | 1 | (1.2) |
| 4 | 0 | (0.0) | 0 | (0.0) | 0 | (0.0) | 0 | (0.0) |

| | | |
|---|---|---|
| P-value [3] | <0.0001 (S) | <0.0001 (S) |

^{[1]}If ACC count = 0 then Grade = 0; if count = 1-10 then Grade = 1; if count = 11-20 then Grade = 2; if count = 21-50 then Grade = 3; if count >50 then Grade = 4.
^{[2]}Subjects with ACC grades equal to 0 and who received rescue medication on or before the assessment day 15 are counted separately from subjects who did not receive rescue medication.
[3] P-values are from a Chi-square test for association between treatment and responder status. A subject with an ACC grade of 0 at the last assessment on or prior to the Day 15 visit, and did not take rescue medication, is considered a responder to therapy. Any subject that has an ACC score of >0 at the last assessment on or prior to the Day 15 visit, or took rescue medication, is considered a non-responder

It was observed that more than 50% of subjects had an anterior chamber cell (ACC) grade of zero (0) on day 15.

Key Secondary Efficacy Endpoint: Proportion of Subjects with Score of Zero (0) on Visual Analog Scale (VAS) at Day 15

A comparison of the proportion of subjects with a VAS score of Zero (0) showed statistically significant results (p-values=0.0006) favouring Test versus Reference groups for both the intent-to-treat (ITT) population and the Per Protocol (PP) population. Therefore, the key secondary objective of the study was met.

TABLE 12
Observations

| | mITT Population | | | | PP Population | | |
|---|---|---|---|---|---|---|---|
| | Test Product | | Reference | | Test Product | | Reference |
| Visual | (N = 154) | | (N = 144) | | (N = 123) | | (N = 83) |
| Analog Scale | n (%) | | n (%) | | n (%) | | n (%) |
| 0 (No Pain) | 145 | (94.2) | 79 | (54.9) | 122 | (99.2) | 72 (86.7) |
| >0 | 2 | (1.3) | 11 | (7.6) | 1 | (0.8) | 10 (12.0) |

| | | |
|---|---|---|
| P-value [1] | 0.0006 (S) | 0.0005 (S) |

Note:
OC (The observed cases): The missing data will not be imputed in this approach, only observed data will be considered.
Note 2: Note:
Eye pain/discomfort was evaluated at every visit using a Visual Analog Scale (VAS), scoring from 0 to 100 using a mark on a 100 mm line (0 = absent; 100 = maximum)
[1] Difference between the treatment groups at Day 15 was tested using a Fisher's exact test at the 0.05 significance level.

It was observed that more than 90% of subjects had a pain score of zero (0) on visual analog scale at day 15.

Summary of Treatment Emergent Adverse Events (TEAEs)

There were a total of 508 TEAEs, 317 in the Reference group and 191 in Test group. These TEAEs occurred in 87 Test group subjects (56.5%) and 96 Reference group subjects (66.2%). Only 4 subjects (2 in each group) experienced Serious TEAEs. None of them was related to study drug. Twelve (7.8%) subjects experienced drug related TEAEs in the Test group, and 25 (17.2%) in the Reference group. Study drug was permanently discontinued in 24 (8.0%) subjects due to TEAEs. A total of 63 subjects received concomitant medications as an action taken for the TEAEs. TEAEs resolved in 157 (52.5%) subjects, while for 45 (15.1%) subjects the events were ongoing at study end.

TABLE 13
Observations

| | Test Product | | Reference | | Total | |
|---|---|---|---|---|---|---|
| Overall Incidence | (N = 154) | | (N = 145) | | (N = 299) | |
| Overall Incidence of TEAEs | 87 | (56.5) | 96 | (66.2) | 183 | (61.2) |
| Overall Frequency of TEAEs | 191 | | 317 | | 508 | |
| Overall Incidence of Serious | 2 | (1.3) | 2 | (1.4) | 4 | (1.3) |
| TEAEs | | | | | | |
| Study Drug Related TEAEs | 12 | (7.8) | 25 | (17.2) | 37 | (12.4) |
| Study Drug Related Serious | 0 | (0.0) | 0 | (0.0) | 0 | (0.0) |
| TEAEs | | | | | | |

TEAEs by Highest Severity

| | | | | | | |
|---|---|---|---|---|---|---|
| Mild | 60 | (39.0) | 33 | (22.8) | 93 | (31.1) |
| Moderate | 26 | (16.9) | 47 | (32.4) | 73 | (24.4) |
| Severe | 1 | (0.6) | 16 | (11.0) | 17 | (5.7) |

Related TEAEs by Highest Relationship to Treatment

| | | | | | | |
|---|---|---|---|---|---|---|
| Possibly | 9 | (5.8) | 11 | (7.6) | 20 | (6.7) |
| Probably | 2 | (1.3) | 10 | (6.9) | 12 | (4.0) |
| Certainly | 1 | (0.6) | 4 | (2.8) | 5 | (1.7) |

Action Taken for Subjects with TEAEs

| | | | | | | |
|---|---|---|---|---|---|---|
| None | 67 | (43.5) | 76 | (52.4) | 143 | (47.8) |
| Study drug permanently | 2 | (1.3) | 22 | (15.2) | 24 | (8.0) |
| discontinued/withdrawn | | | | | | |
| Not Applicable | 38 | (24.7) | 34 | (23.4) | 72 | (24.1) |

Other Action Taken for Subjects with TEAEs

| | | | | | | |
|---|---|---|---|---|---|---|
| None | 74 | (48.1) | 85 | (58.6) | 159 | (53.2) |
| Concomitant Medication taken | 25 | (16.2) | 38 | (26.2) | 63 | (21.1) |
| Required procedure | 3 | (1.9) | 0 | (0.0) | 3 | (1.0) |
| Other | 1 | (0.6) | 1 | (0.7) | 2 | (0.7) |

Outcome for Subjects with TEAEs

| | | | | | | |
|---|---|---|---|---|---|---|
| Resolved | 81 | (52.6) | 76 | (52.4) | 157 | (52.5) |
| Resolving | 5 | (3.2) | 11 | (7.6) | 16 | (5.4) |
| Not Resolved | 12 | (7.8) | 17 | (11.7) | 29 | (9.7) |
| Unknown | 0 | (0.0) | 1 | (0.7) | 1 | (0.3) |

NOTE:
Percentages are based on the Safety Population.

It was found by the present inventors that the difluprednate ophthalmic aqueous solution of the present disclosure having 0.04% w/v difluprednate when administered twice-a-day into the eye of the subject for treating pain and inflammation post ocular surgery, lead to the subjects having an anterior chamber cell (ACC) grade of 0 and a pain score of 0 on the visual analog scale (VAS), wherein the subject has not received rescue medication.

I/We claim:

1. A method of reducing inflammation in the anterior chamber of the eye comprising administering into the eye of a subject that has undergone ocular surgery an aqueous solution comprising difluprednate as the sole active ingredient at a concentration of 0.02% to 0.04% weight by volume in an aqueous vehicle,
wherein the solution is free of oil,
wherein the solution is administered twice-a-day,
wherein the subject has not received rescue medication, and
wherein after the treatment the subject has an anterior chamber cell (ACC) grade of 0 and a pain score of 0 on the visual analog scale (VAS) post ocular surgery following 15 days of administration.

2. The method as claimed in claim 1, wherein the ocular surgery is cataract surgery.

3. A method of achieving an anterior chamber cell (ACC) grade of 0 in a subject in need thereof, the method comprising administering into the eye of said subject an aqueous solution comprising difluprednate as the sole active ingredient at a concentration of 0.02% to 0.04% weight by volume in an aqueous vehicle,
wherein the solution is free of oil,
wherein the solution is administered twice-a-day for 7-21 days, and
wherein the subject has not received rescue medication.

4. The method as claimed in claim 3, wherein the subject in need thereof has undergone ocular surgery.

5. A method of achieving a pain score of 0 on the visual analog scale (VAS) in a subject in need thereof, the method comprising administering into the eye of said subject an aqueous solution comprising difluprednate as the sole active ingredient at a concentration of 0.02% to 0.04% weight by volume in an aqueous vehicle,
wherein the solution is free of oil,
wherein the solution is administered twice-a-day for 7-21 days, and
wherein the subject has not received rescue medication.

6. The method as claimed in claim 5, wherein the subject in need thereof has undergone ocular surgery.

7. A method of treatment of inflammatory disorder of eye, said method comprising administering into the eye of a patient in need thereof an aqueous solution comprising difluprednate as the sole active ingredient at a concentration of 0.02% to 0.04% w/v in an aqueous vehicle, wherein the solution is free of oil and wherein the solution is administered twice-a-day for 14 days, wherein the patient has not received rescue medication, and after the treatment the patient has an anterior chamber cell grade of 0.

8. The method as claimed in claim 7, wherein the inflammatory disorder of the eye is pain and inflammation associated with ocular surgery.

9. The method as claimed in claim 8, wherein the ocular surgery is cataract surgery.

10. The method as claimed in claim 7, wherein the difluprednate is present at a concentration of 0.04% weight by volume.

11. The method as claimed in claim 7, wherein the method results in a pain score of 0 on the visual analog scale at day 15 post ocular surgery.

12. The method as claimed in claim 11, wherein the pain score on visual analog scale is evaluated based on eye pain and discomfort scoring from 0 to 100.

13. An aqueous solution comprising difluprednate as the sole active ingredient at a concentration of 0.02% to 0.04% weight by volume in an aqueous vehicle for use in the reduction of inflammation in the anterior chamber of the eye of a subject that has undergone ocular surgery,
wherein the solution is free of oil,
wherein the solution is administered twice-a-day,
wherein the subject has not received rescue medication, and
wherein after the treatment the subject has an anterior chamber cell (ACC) grade of 0 and a pain score of 0 on the visual analog scale (VAS) post ocular surgery following 15 days of administration.

14. The aqueous solution for use in the reduction of inflammation in the anterior chamber of the eye as claimed in claim 13, wherein the ocular surgery is cataract surgery.

15. An aqueous solution comprising difluprednate as the sole active ingredient at a concentration of 0.02% to 0.04% weight by volume in an aqueous vehicle for use in achieving an anterior chamber cell (ACC) grade of 0 in a subject in need thereof,
wherein the solution is free of oil,
wherein the solution is administered twice-a-day for 7-21 days, and
wherein the subject has not received rescue medication.

16. The aqueous solution for use in the reduction of inflammation in the anterior chamber of the eye as claimed in claim 15, wherein the subject in need thereof has undergone ocular surgery.

17. An aqueous solution comprising difluprednate as the sole active ingredient at a concentration of 0.02% to 0.04% weight by volume in an aqueous vehicle for use in achieving a pain score of 0 on the visual analog scale (VAS) in a subject in need thereof,
wherein the solution is free of oil,
wherein the solution is administered twice-a-day for 7-21 days, and
wherein the subject has not received rescue medication.

18. The aqueous solution for use in the reduction of inflammation in the anterior chamber of the eye as claimed in claim 17, wherein the subject has undergone ocular surgery.

19. An aqueous solution comprising difluprednate as the sole active ingredient at a concentration of 0.02% to 0.04% w/v in an aqueous vehicle for use in the treatment of inflammatory disorder of eye, wherein the solution is free of oil and wherein the solution is administered twice-a-day for 14 days, wherein the patient has not received rescue medication, and after the treatment the patient has an anterior chamber cell grade of 0.

20. The aqueous solution for use in the treatment of inflammatory disorder of eye as claimed in claim 19, wherein the inflammatory disorder of the eye is pain and inflammation associated with ocular surgery.

21. The aqueous solution for use in the treatment of inflammatory disorder of eye as claimed in claim 20, wherein the ocular surgery is cataract surgery.

22. The aqueous solution for use in the treatment of inflammatory disorder of eye as claimed in claim 19, wherein the difluprednate is present at a concentration of 0.04% weight by volume.

23. The aqueous solution for use in the treatment of inflammatory disorder of eye as claimed in claim 19, wherein the treatment results in a pain score of 0 on the visual analog scale at day 15 post ocular surgery.

24. The aqueous solution for use in the treatment of inflammatory disorder of eye as claimed in claim 23, wherein the pain score on visual analog scale is evaluated based on eye pain and discomfort scoring from 0 to 100.
